(19) United States
(12) Patent Application Publication
(10) Pub. No.: US 2015/0100116 A1
(43) Pub. Date: Apr. 9, 2015

(54) IMPLANT AND METHOD FOR IMPROVING COAPTATION OF AN ATRIOVENTRICULAR VALVE

(71) Applicant: Medizinische Universitat Wien, Wien (AT)

(72) Inventors: Werner MOHL, Altenmarkt/Thennenberg (AT); Werner REICHENFELSER, Wien (AT)

(73) Assignee: Medizinische Universitat Wien, Wien (AT)

(21) Appl. No.: 14/047,920

(22) Filed: Oct. 7, 2013

(51) Int. Cl. A61F 2/24

(52) U.S. Cl. CPC A61F 2/2403; A61F 2/243
USPC 623/2.11; 623/2.17

(57) ABSTRACT

The invention relates to an implant and a method for improving coaptation of an atrioventricular valve, the atrioventricular valve having a native first leaflet, a native second leaflet and an annulus. The implant comprises a support structure and a flexible artificial leaflet structure mounted to the support structure and shaped to coapt with the native second leaflet.

### FIELD

The invention relates to an implant and a method for improving coaptation of an atrioventricular valve.

### BACKGROUND

Atrioventricular valves are membraneous folds that prevent backflow from the ventricles of the human heart into the atrium during systole. They are anchored within the ventricular cavity by chordae tendineae, which prevent the valve from prolapsing into the atrium.

The chordae tendineae are attached to papillary muscles that cause tension to better hold the valve. Together, the papillary muscles and the chordae tendineae are known as the subvalvular apparatus. The function of the subvalvular apparatus is to keep the valves from prolapsing into the atria when they close. The opening and closure of the valves is caused by the pressure gradient across the valve.

The human heart comprises two atrioventricular valves, the mitral valve and the tricuspid valve. The mitral valve allows the blood to flow from the left atrium into the left ventricle. The tricuspid valve is located between the right atrium and the right ventricle. The mitral valve has two leaflets that are each divided into several scallops: the anterior leaflet has three scallops (A1,A2,A3), the posterior leaflet has three scallops (P1,P2,P3). The tricuspid valve has three leaflets. Engagement of corresponding surfaces of the leaflets against each other is decisive for providing closure of the valve to prevent blood flowing in the wrong direction. The closure forms a so called coaptation area.

Native heart valves become dysfunctional for a variety of pathological causes. Failure of the leaflets to seal during ventricular systole is known as malcoaptation, and may allow blood to flow backward through the valve (regurgitation). Malcoaptation is often caused by a dilatation of the annulus. Another reason is a restriction in motion or an excessive motion of the leaflet structures. Heart valve regurgitation can result in cardiac failure, decreased blood flow, lower blood pressure, and/or a diminished flow of oxygen to the tissues of the body. Mitral regurgitation can also cause blood to flow back from the left atrium to the pulmonary veins, causing congestion and backward failure.

Some pathologies of atrioventricular valves, such as malcoaptation, often require reconstruction of the valvular and subvalvular apparatus as well as redesigning the enlarged annulus. Sometimes a complete surgical replacement of the natural heart valve with heart valve prosthesis is necessary. There are two main types of artificial heart valves: the mechanical and the biological valves. The mechanical-type heart valve uses a pivoting mechanical closure supported by a base structure to provide unidirectional blood flow. The tissue-type valves have flexible leaflets supported by a base structure and projecting into the flow stream that function similar to those of a natural human heart valve and imitate their natural flexing action to coapt against each other. Usually two or more flexible leaflets are mounted within a peripheral support structure made of a metallic or polymeric material. In transcatheter implantation the support within the annulus may be in the form of a stent, as is disclosed in US 2011/0208298 A1.

In order to provide enough space for the artificial leaflets to work properly, the peripheral support is positioned in the native valve so as to force the native leaflets apart. To this end and in order to provide appropriate anchoring of the peripheral support within the native valve, the same is fixed to the native leaflets by suitable means. However, in some applications, such as with mitral valves, fixing the peripheral support to the native anterior leaflet and dislocating the same from its natural position may cause an obstruction of the outflow tract and of the aortic valve, which is located in the left ventricle immediately adjacent the anterior leaflet.

The gold standard for treating mitral regurgitation is to repair the mitral apparatus including leaflets and the subvalvular apparatus and to reshape the mitral annulus (Carpentier technique). If repair is not possible an excision of the valve including parts of the subvalvular apparatus is performed with subsequent implantation of a heart valve prosthesis. This is necessary particularly when the valve is destructed by inflammation. Although in most instances a complete excision of the destroyed valve is necessary, sometimes a partial replacement is possible. A clinically used mitral valve restoration system (Mitrofix®) replaces only the posterior leaflet with a rigid prosthesis mimicking a fixed posterior leaflet allowing the natural anterior leaflet to coapt. This prosthesis is also sewn into the position of the destroyed posterior aspect of the annulus. This requires open heart surgery and extended cardiac arrest.

Recent trends focus on less invasive procedures to minimize surgical trauma and to perform transcatheter approaches including transatrial, transaortal or transapical procedures to replace or reconstruct dysfunctional valves thus minimizing the need of or avoiding heart lung machine and cardiac arrest. Whereas this is a common procedure in aortic valves nowadays, only few mitral valves insufficiencies are corrected by percutaneous or transapical procedures. Most of these concepts are redesigning and remodeling artificially the mitral annulus to allow coaptation or to enforce coaptation by fixing both leaflets together with a clip reducing mitral regurgitant flow. Percutaneously or transapically deployed valve prostheses are difficult to anchor due to the special anatomy of the mitral valve and the vicinity of the anterior leaflet to the aortic outflow tract.

### SUMMARY

Therefore, it is an object of the instant invention to provide an improved implant for improving coaptation of an atrioventricular valve. In particular, it is an object of the invention to provide an implant that does not involve the risk of stenosis of the aortic valve.

It is a further object of the invention to provide an implant that can be easily deployed to the target site.

It is a further object of the invention to use preoperative imaging data to construct a posterior leaflet according to the patient's pathologic anatomy.

The invention generally provides improved medical implants and methods for the treatment of regurgitation in atrioventricular valves, in particular mitral valves. In some embodiments, the invention provides a medical implant that provides replacement of one of the two or three native leaflet parts of atrioventricular valves, while leaving the other native leaflet(s) fully functional. In case of an implant configured for mitral valves, the medical implant preferably provides replacement of the native posterior leaflet, while leaving the native anterior leaflet fully functional. Preferably, the implant does not comprise any structure that is fixed to the anterior leaflet. When configured for the mitral valve, the implant preferably affects only one half of the valve, and only extends over the region of the posterior leaflet.

In the context of the instant invention, the terms “replacement” and “replacing” mean that the artificial leaflet replaces the function of a damaged or otherwise malfunctional native leaflet. However, the damaged or otherwise malfunctional native leaflet is not physically removed. Rather, the damaged or otherwise malfunctional native leaflet is left in the valve. The damaged or otherwise malfunctional native leaflet may be at least partially displaced by the artificial leaflet of the invention. Further, and the damaged or otherwise malfunctional native leaflet may support the function of the artificial leaflet.

In some embodiments, the artificial leaflet is flexible in order to allow the artificial leaflet to behave like the artificial leaflet it replaces. In particular, the artificial is flexible at least in its lower end region, i.e. the end region facing the ventricular cavity.

In some embodiments, the invention provides an implant for improving coaptation of an atrioventricular valve, the atrioventricular valve having a native first leaflet, a native second leaflet and an annulus, the implant comprising a support structure and a flexible artificial leaflet structure mounted to the support structure and shaped to coapt with the native second leaflet.

In some embodiments, the invention provides an implant for improving coaptation of an atrioventricular valve, the atrioventricular valve having a native first leaflet, a native second leaflet and an annulus, the annulus having a substantially semicircular first segment, from which the native first leaflet emerges, and a substantially semicircular second segment, from which the native second leaflet emerges, the implant comprising a support structure and an artificial leaflet structure mounted to the support structure and shaped to coapt with the native second leaflet, said support structure being anchored only to the first segment of the annulus.

In case of an implant configured for mitral valves, the first native leaflet is a posterior leaflet of the mitral valve and the second native leaflet is an anterior leaflet of the mitral valve. The artificial leaflet is configured as an artificial posterior leaflet and replaces and/or supports the function of the native posterior leaflet. The artificial posterior leaflet is preferably shaped such as to improve coaptation with the native anterior leaflet.

In case of an implant configured for tricuspid valves, the first native leaflet is an anterior leaflet of the tricuspid valve and the second native leaflet is a posterior leaflet and the third leaflet is the septal leaflet of the tricuspid valve. The artificial leaflet is configured to replace the function of the native anterior and or posterior leaflet. The artificial anterior or posterior leaflet or the combination of both is preferably shaped such as to improve coaptation with the native anterior and posterior leaflet.

The support structure is configured to carry the artificial leaflet structure and to hold the artificial leaflet structure in a position, in which it can coapt with the native second leaflet. Preferably, the artificial leaflet is held in a position closer to the native second leaflet when compared to the position of the malcoapting native first leaflet. In particular, the artificial leaflet bears against the native second leaflet and, depending on the degree of pathological dilatation of the annulus, displaces the native first leaflet to a location closer to the wall of the ventricle when compared to its original location.

In order to associate the implant to the annulus, the support structure preferably comprises an upper support element and a lower support element displaceable relative to each other so as to be able to squeeze a section of the annulus between them in order to avoid improper paravalvular leakage and regurgitation.

The upper support element preferably is substantially U-shaped, semicircular or circular so as to conform to the shape of the annulus or a section of the annulus. In order to stabilize the upper support element, the upper support element preferably comprises bracing means for applying a radial bracing force across the annulus and the adjacent atrial wall. The bracing force acts so as to spread apart the annulus, so as to firmly hold the upper support element relative to the annulus.

In some embodiments of the invention, the upper support element extends only over the first segment of the annulus.

Fixing the support structure relative to the annulus preferably comprises arranging the upper support element at least partially within the inner circumferential surface of the annulus and expanding the upper support element in a radial direction towards the inner circumferential surface of the annulus.

In order to enable an expansion of the upper support element so as to apply said bracing force, the support structure preferably comprises a cavity. The upper support element is preferably expanded by filing a filling material into a cavity. The filling material may be selected from the group consisting of a fluid, an elastic solid, such as a foamed material, and a gel. The cavity preferably comprises a closable opening for filling the cavity with the filling material. The filling material is preferably filled into the cavity after the implant has been deployed to the heart. Alternatively, the upper support element is expanded by expanding a filling material contained in the cavity. In this case, the filling material may be already present in the cavity before the implant is deployed to the heart. The filling material may be a liquid that forms a foamed structure as soon as a chemical reaction is initiated by applying heat, radiation, water or the like.

Further, the lower support element of the support structure preferably comprises a cavity. The lower support element is preferably expanded by filing a filling material into a cavity. The filling material may be selected from the group consisting of a fluid, an elastic solid, such as a foamed material, and a gel. The cavity preferably comprises a closable opening for filling the cavity with the filling material. The filling material is preferably filled into the cavity after the implant has been deployed to the heart. Alternatively, the lower support element is expanded by expanding a filling material contained in the cavity. In this case, the filling material may be already present in the cavity before the implant is deployed to the heart. The filling material may be a liquid that forms a foamed structure as soon as a chemical reaction is initiated by applying heat, radiation, water or the like.

Due to the expansion of the upper support element and/or the lower support element the annulus can be effectively squeezed between the upper and the lower support element.

According to another preferred embodiment, the artificial leaflet structure comprises a cavity. The closed cavity contains or may be filled with a filling material so as to expand to a defined shape and volume. Once expanded, the artificial leaflet structure has an increased structural stability and may adopt a defined surface shape that improves coaptation with the native second leaflet. The artificial leaflet structure may comprise several cavities that are connected with each other. The filling material may be selected from the group consisting of a fluid, an elastic solid, such as a foamed material, and a gel. The cavity preferably comprises a closable opening for filling the cavity with the filling material. The filling material is preferably filled into the cavity after the implant has been deployed to the heart. Alternatively, the artificial leaflet is expanded by expanding a filling material contained in the cavity. In this case, the filling material may be already present in the cavity before the implant is deployed to the heart. The filling material may be a liquid, that forms a foamed structure as soon as a chemical reaction is initiated by applying heat, radiation, water or the like. In some embodiments the filled semiflexible material is sculptured by the mechanical force of the second leaflet within the first closing attempts until the filled material receives its permanent shape.

Preferably, the cavity of the artificial leaflet structure and the cavity of the support structure are connected to each other to form a single cavity.

In some embodiments, the invention provides an implant for improving coaptation of an atrioventricular valve, the implant comprising a support structure and a flexible artificial leaflet structure mounted to the support structure and shaped to coapt with the native second leaflet, wherein the support structure and the artificial leaflet structure are deployable from a first position, in which the support structure and the artificial leaflet structure are arranged within the tubular housing, into a second position, in which the artificial leaflet structure is deployed to coapt with the second native leaflet. In this way, the implant can be easily deployed to the heart by minimal invasive surgery. In particular, the tubular housing is preferably advanced into the heart by means of a catheter transatrially, transseptally, transfemorally or transapically.

Preferably, the support structure and the artificial leaflet structure are configured to be deployed from a folded or rolled-up state into an extended state. In the folded or rolled-up state, the structures may easily be advanced to the heart transcatheterally.

The artificial leaflet may be made of a biocompatible material, such as polyethylene or polyurethane, polyfluorethylen (Goretex®) or from natural tissue such as heterologic pericardium.

The support structure preferably comprises a wire of a memory-shape material, such as Nitinol.

Preferably, the implant further comprises retention means connected to the support structure and the artificial leaflet for preventing prolapse of the artificial leaflet.

According to a further aspect the invention refers to a method of improving coaptation of an atrioventricular valve, the atrioventricular valve having an annulus, a native first leaflet and a native second leaflet, the method comprising:
providing an implant comprising a support structure and a flexible artificial leaflet structure mounted to the support structure, the implant being arranged in a tubular housing,
advancing the tubular housing by means of a catheter through a body vessel of a patient into the heart,
deploying the implant from the tubular housing,
fixing the support structure relative to the annulus,
arranging the artificial leaflet structure adjacent the native first leaflet such that the artificial leaflet structure can coapt with the native second leaflet.

Preferably, the native first leaflet is a native posterior leaflet of a mitral valve and the second native leaflet is an anterior leaflet of the mitral valve. The artificial leaflet is configured as an artificial posterior leaflet and replaces the normal function of the native posterior leaflet. The artificial posterior leaflet is preferably shaped such as to improve coaptation with the native anterior leaflet.

Preferably, the tubular housing is advanced into the heart by means of a catheter transatrially, i.e. through the left atrium of the heart, transseptally, i.e. through the septum of the heart, transfemorally or transapically, i.e. through the apex of the heart. The positioning is facilitated by a steerable guiding element to maneuver the deployable element into the rim of the annulus connecting the ventricular wall with the leaflet structure.

Preferably, the step of fixing the support structure relative to the annulus comprises positioning an upper support element on a superior surface of the annulus and positioning a lower support element on an inferior surface of the annulus thereby clamping a section of the annulus between the upper support element and the lower support element.

Preferably, the step of fixing the support structure relative to the annulus comprises arranging the upper support element at least partially within the inner circumferential surface of the annulus and expanding the upper support element in a radial direction towards the inner circumferential surface of the annulus.

Preferably, the upper support element is expanded by filling a filling material into a cavity of the upper support element.

Preferably, the upper support element is expanded by expanding a filling material arranged in a cavity of the upper support element.

Preferably, the lower support element is expanded by filling a filling material into a cavity of the lower support element.

Preferably, the lower support element is expanded by expanding a filling material arranged in a cavity of the lower support element.

Preferably, the method further comprises connecting the artificial leaflet to the support structure by the aid of retention means for preventing prolapse of the artificial leaflet.

In some embodiments, the invention provides a method comprising the steps of
imaging the native mitral valve prior to the procedure,
identifying and localizing the areas of malcoaptation,
measuring leaflet heights in all three scallops (p1,p2,p3) and their form and the two indentations,
measuring the extend of the posterior leaflet,
virtual reconstructing of an artificial posterior leaflet with scallops and artificial chordae,
implementing the patient's mitral valve into a computer model, thereby obtaining 3D data of the mitral valve,
adapting the 3D data in the computer model to improve coaptation,
using the adapted 3D data from the computer model to obtain 3D data representative of the three scallops as well as of the wall coverage of the posterior leaflet,
3D printing of artificial scallops of the posterior leaflet from said 3D data,
using the artificial scallops as a model and building an artificial posterior leaflet on said model, optionally including modeling cushion sizes and forms for the definite coaptation surface area,
connecting the artificial posteriori leaflet to a support structure,
folding the support structure and the artificial leaflet and arranging the same into a tubular housing,
delivering the tubular housing by means of a catheter transatrially, transseptally, transfemorally or transapically to the mitral valve of the heart,
anchoring the support structure to the native mitral valve.

### BRIEF DESCRIPTION OF THE DRAWINGS

FIG. 1 is a schematic illustration of a human heart,

FIGS. 2-8 are schematic illustrations of the consecutive steps of deploying a mitral valve implant in a first embodiment,

FIG. 9 is a schematic illustration of a second embodiment of a mitral valve,

FIG. 10 is a schematic illustration of an alternative way of a mitral valve implant deployment,

FIG. 11 is a schematic illustration of the first embodiment of the mitral valve folded so as to be deployable by means of a catheter,

FIG. 12 is a top view of the first embodiment of the mitral valve in a deployed condition,

FIG. 13 is a side view of the first embodiment of the mitral valve in a deployed condition,

FIGS. 14-19 are side views of the first embodiment of the mitral valve in different steps of the deployment procedure.

### DETAILED DESCRIPTION

Aspects of the present invention are disclosed in the following description and related figures directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention.

It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

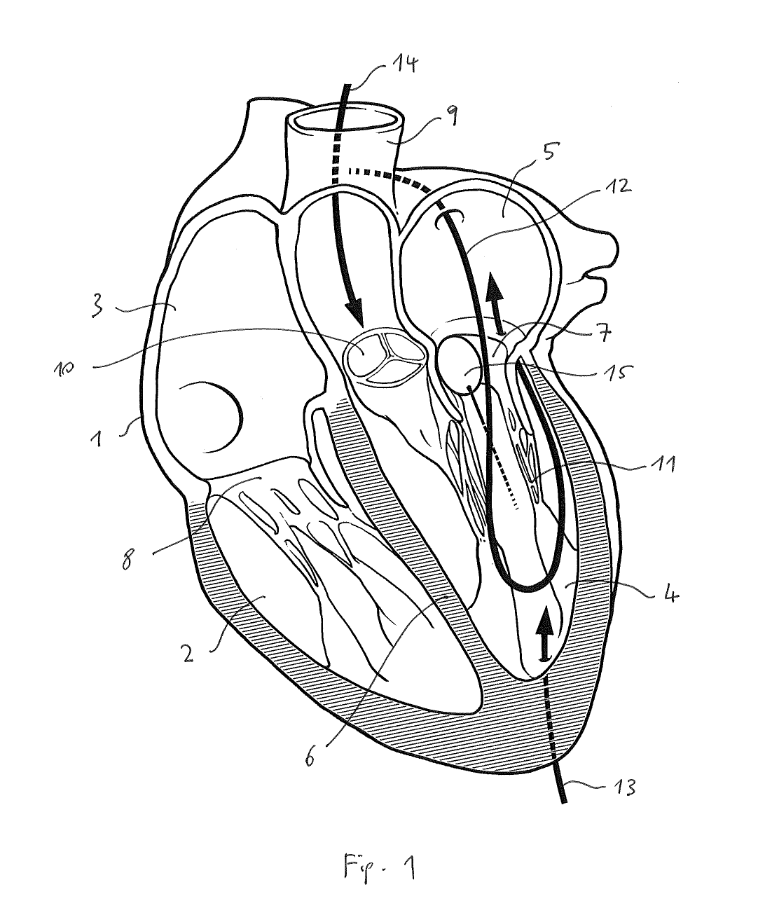

In FIG. 1 is a schematic illustration of a human heart 1 comprising the right ventricle 2, the right atrium 3, the left ventricle 4 and the left atrium 5. The septum 6 divides the heart 1 in a right and a left section. The mitral valve 7 allows the blood to flow from the left atrium 5 into the left ventricle 4. The tricuspid valve 8 is located between the right atrium 3 and the right ventricle 2. The ascending aorta 9 originates at the orifice of the aortic valve 10. The mitral valve 7 comprises an anterior leaflet and a posterior leaflet that are anchored within the left ventricular cavity by chordae tendineae 11, which prevent the valve 7 from prolapsing into the left atrium 5.

The mitral valve implant of the invention is configured to be deployed to the heart transcatheterally. In particular, the implant can be delivered to the heart by means of a catheter transatrially, i.e. through the left atrium of the heart, transseptally, i.e. through the septum 6 of the heart as depicted by line 12, transapically, i.e. through the apex of the heart as depicted by line 13, or through the ascending aorta 9 as depicted by line 14.

During the implant procedure a balloon 15 is placed into the orifice of the mitral valve 7, which is inflated during systole and deflated during diastole to minimize regurgitant volume flow and to prevent severe inflow into the pulmonary veins.

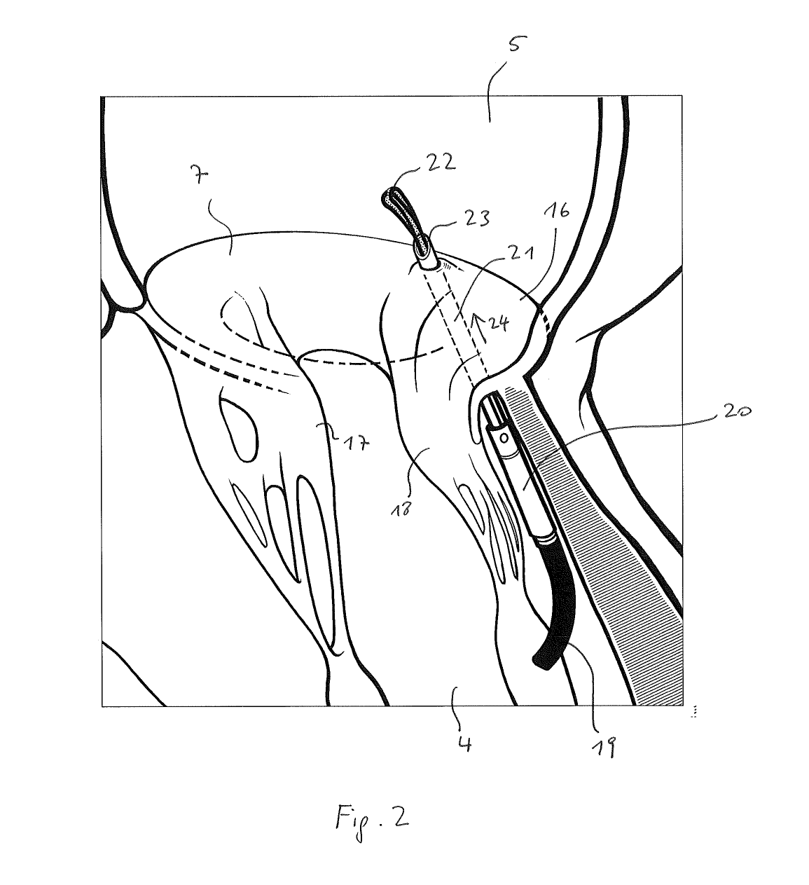

In FIG. 2 the mitral valve 7 is shown in more detail. The mitral valve 7 comprises an annulus 16, from which the anterior leaflet 17 and the posterior leaflet 18 emerge. In a pathological condition of the mitral valve 7, the annulus 16 can be dilated so that the anterior leaflet 17 and the posterior leaflet 18 fail to coapt and do not provide a tight seal between the left ventricle 4 and the left atrium 5 during systole.

The catheter to deliver the implant to the heart is denoted with reference number 19 and carries a tubular housing 20 on its free end, in which the implant is arranged in a compacted, in particular folded state during delivery. The catheter 19 comprises an inner movable member 21 in the form of a hollow cylinder. The inner movable member 21 is guided to be movable in an axial direction relative to the housing 20 and comprises a chamfered tip 23. As can be seen in FIG. 2 the inner movable member 21 has been advanced in the direction or arrow 24 to penetrate the annulus 16 from below, i.e. from the left ventricle 4, so that the tip 23 of the inner movable member 21 protrudes into the left atrium 5. The position of the penetration point preferably is arranged between the two papillary muscles of the subvalvular apparatus of the posterior leaflet. To find the exact penetration position, the positioning of the chamfered tip 23 is facilitated by a steerable catheter element with electrodes.

The inner movable member 21 has an opening at its distal end in order to deploy the implant to the implantation site. In FIG. 2 a part of the upper support element 22 of the implant projects from the movable member 21.

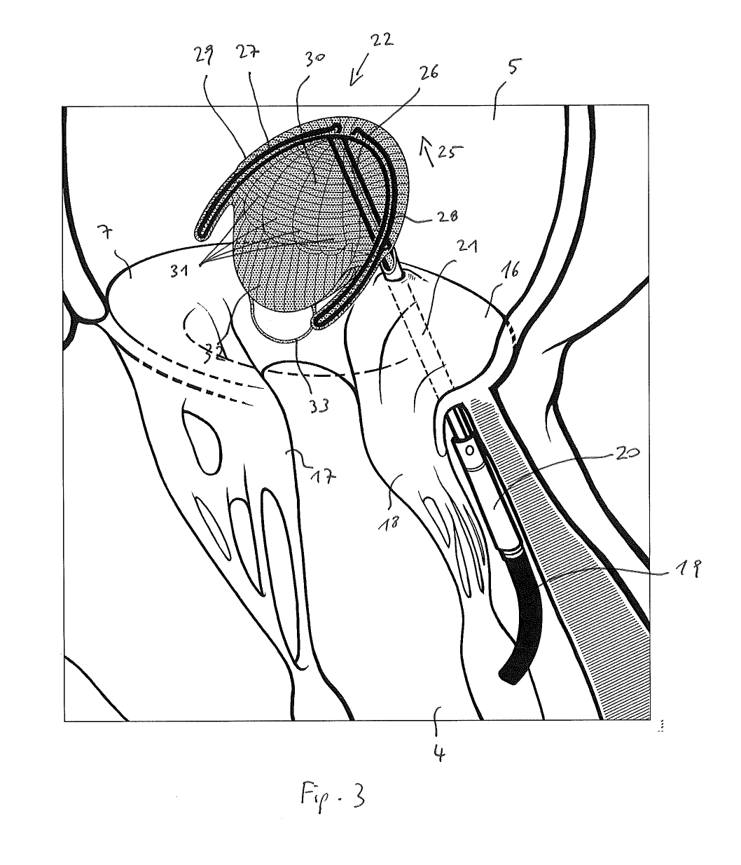

FIG. 3 illustrates the deployment of the upper support element 22 of the support structure. The upper support element 22 has been pushed forward according to arrow 25 so that it completely exits the movable member 21. The upper support element 22 comprises a straight base section 26 and side arms 27 and 28. The side arms 27,28 and the base section 26 are made from at least one wire, wherein a memory-shape material, such as Nitinol is preferred. When housed in the inner movable member 21, the side arms 27 and 28 are folded down and extend parallel to the straight base section 26. Once deployed from the inner movable member 26, the side arms 27,28 fold out to the side and up, so that they come to lie in a common plane that encloses an angle α of 70-90° with the straight base section 26.

The arms 27,28 are shaped to substantially conform to the curvature of the annulus 16. In the embodiment according to FIGS. 2 to 8 the arms 27,28 extend only over a part of the circumference of annulus 16. In particular, the arms 27,28 of the upper support element extend only over the segment of the annulus 16, from which the posterior leaflet 18 emerges.

The arms 27,28 of the upper support element 22 are received in a cavity of a jacket 29 surrounding the arms 27,28. The jacket 29 is integral with an artificial leaflet 30 and is made of a biocompatible material, such as polyethylene or polyurethane, polyfluorethylen (Goretex®) or from natural tissue such as heterologic pericardium. The artificial leaflet comprises a first section immediately adjacent the jacket 29, in which the artificial leaflet 30 comprises a plurality of cushion-like embossments 31 mimicking the natural shape of the scallops (p1,p2,p3) of the native posterior leaflet 18. Further, the artificial leaflet 30 comprises an inferior section 32 that is planar and does not comprise a cavity. Further, the inferior section 32 carries a strap 33 that will be described later in more detail.

Turning now to FIG. 4, the movable member 21 together with the upper support element 22 has been retracted according to arrow 34 so that the tip 23 of the movable member 21 is positioned below the annulus 16 and the upper support element 22 is seated against the upper surface of the annulus 16. In doing so, the straight section 26 of the upper support element 22 is retracted with such a pulling force that the angle between the common plane of the arms 27,28 and the straight base enlarged to approximately 90°. Thereby, a constant pre-load is applied onto the upper surface of the annulus 16. Upon retraction of the upper support element 22 the artificial leaflet 30 is seated onto the native posterior leaflet 18.

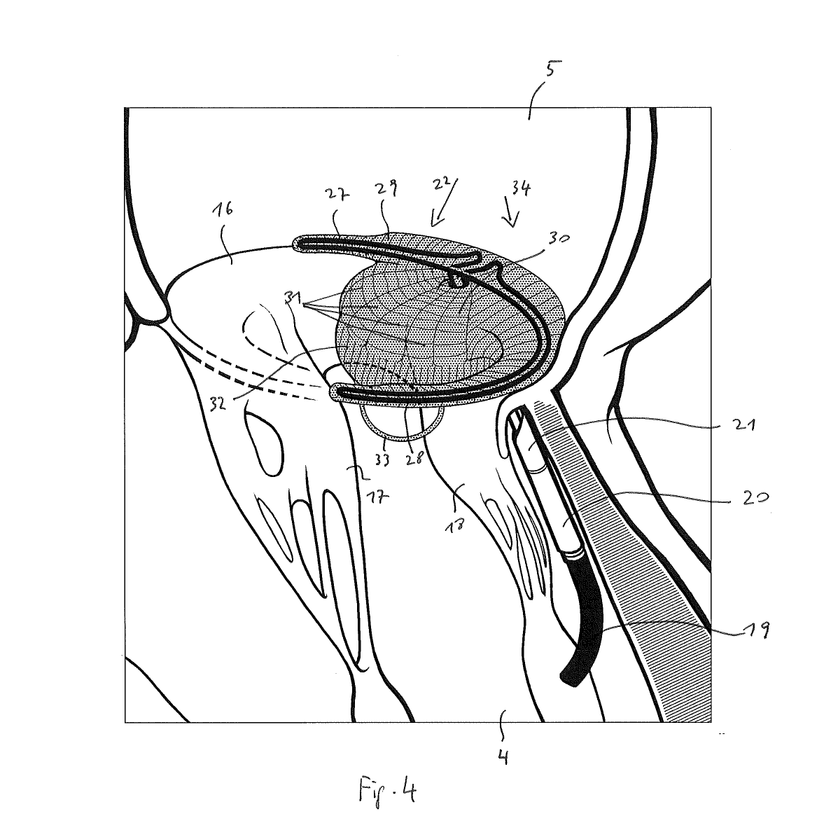

In the illustration according to FIG. 5 the lower support element 35 has been deployed from the movable member 21 via the distal opening of the same. The lower support element 35 comprises two arms 36,37 that have been folded to the side and up, so that they come to lie in a common plane and get seated to the lower surface of the annulus 16, i.e. the surface of the annulus 16 that faces the left ventricle 4. The arms 36,37 are shaped to substantially conform to the curvature of the annulus 16. In the embodiment according to FIGS. 2 to 8 the arms 36,37 extend only over a part of the circumference of annulus 16. In particular, the arms 36,37 of the lower support element 35 extend only over the segment of the annulus 16, from which the posterior leaflet 18 emerges.

The arms 36,37 of the lower support element 35 are received in a cavity of a jacket 38 surrounding the arms 36,37.

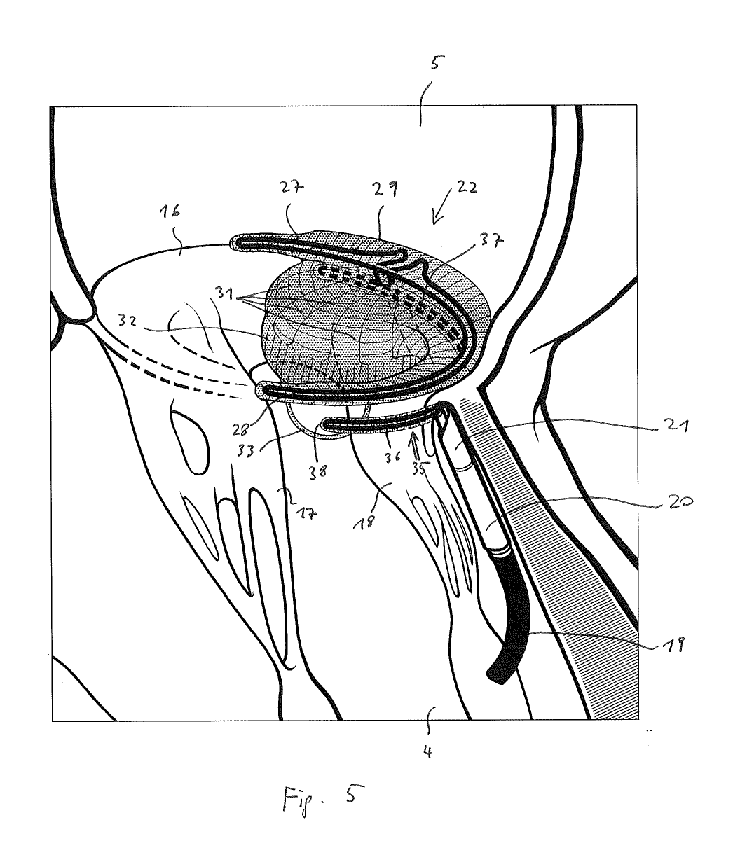

FIG. 6 corresponds to the FIG. 5, but the jackets 29 and 38 as well as the first section of the artificial leaflet 30 (comprising the cushion-like embossments 31) have been “inflated” or expanded. In doing so the annulus 16 is squeezed from above and from below between the jacket 29 and the jacket 38 thereby fixing the position of the support structure. Further, the inflation of the jacket 29 results in a radial expansion along the arms 27,28 so that a radical bracing force is achieved between the outer circumference of the jacket 29 and an inner circumference of the annulus 16.

The inflation of the first section of the artificial leaflet 30 results in that this section receives a desired 3D-shape including a desired 3D surface shape of the coaptation surface in order to improve coaptation with the native anterior leaflet 17.

The inflation of the jackets 29 and 38 as well as of the first section of the artificial leaflet 30 may be achieved in different ways. As an example, the cavities can be filled with a viscous fluid or a gel. The viscous fluid or the gel can be delivered to the cavities through a lumen of the catheter 19. Alternatively, the cavities can be filled with a pre-polymer before the implant is deployed to the heart and a chemical reaction of the pre-polymer can be induced in-situ so as to produce a foamy or porous structure thereby expanding the volume of the respective cavity. Preferably, the amount of filling material or pre-polymer to be inserted into the cavity is calculated according to the e-module of the filling material and the expected and preferred cushion size.

Particularly preferable is the use of a gel as a filling material for the cavity of the artificial leaflet. The gel allows an adaption of the 3D shape of the artificial leaflet at each closing of the valve. In practice, an optimization of the shape is obtained already a few closing cycles after starting of the operation of the implant. In this way the coaptation of the artificial leaflet with the native anterior leaflet is substantially improved.

The inflation of the artificial leaflet 30 results in a dislocation of the native posterior leaflet 18 such that the native posterior leaflet 18 is moved closer to the wall 41 of the heart.

The cavity of jacket 29 may be separate from the cavity of the artificial leaflet 30. Alternatively, the cavity of the artificial leaflet 30 and the cavity of the jacket 29 may be connected to each other to form a single cavity.

FIG. 7 shows the deployment of a leash-like cord or wire 39. The cord or wire 39 has a hook at its free end, which serves to catch and engage with the strap 33. In this way, the inferior region of the artificial leaflet 30 is held in a position so as to prevent prolapsing of the artificial leaflet 30 into the left atrium 5. Alternatively, the chordae of the native leaflet, if still functioning, may be use to support the artificial leaflet motion and prevent prolapsing of the artificial leaflet 30 into the left atrium 5. Another alternative is to embed are more rigid part into the artificial leaflet to prevent prolapse.

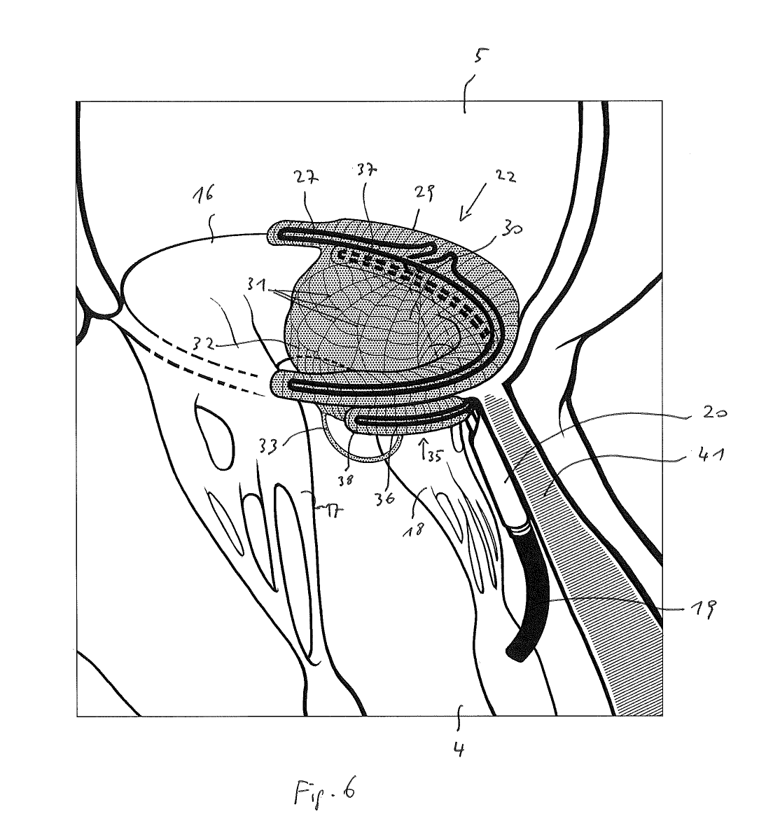

FIG. 8 shows that the degree of retention of the inferior end region of the artificial leaflet 30 can be controlled by varying the length of the cord or wire 39. The length of the cord or wire 39 may be controlled by imaging techniques. In the embodiment shown in FIG. 8, the cord or wire 39 has been completely retracted, so that a maximum of retention force is applied. Further, the catheter 19 has been disconnected form the cylindrical housing 20 of the support structure.

The retention of the inferior end region of the artificial leaflet 30 safeguards the mobility of the anterior leaflet 17 and avoids a systolic anterior movement.

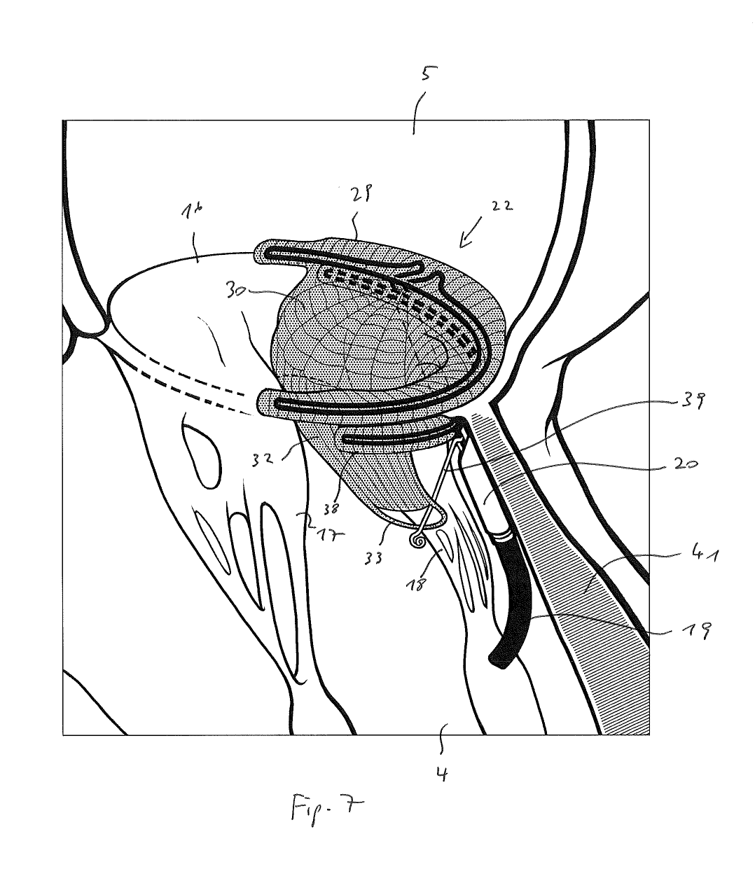

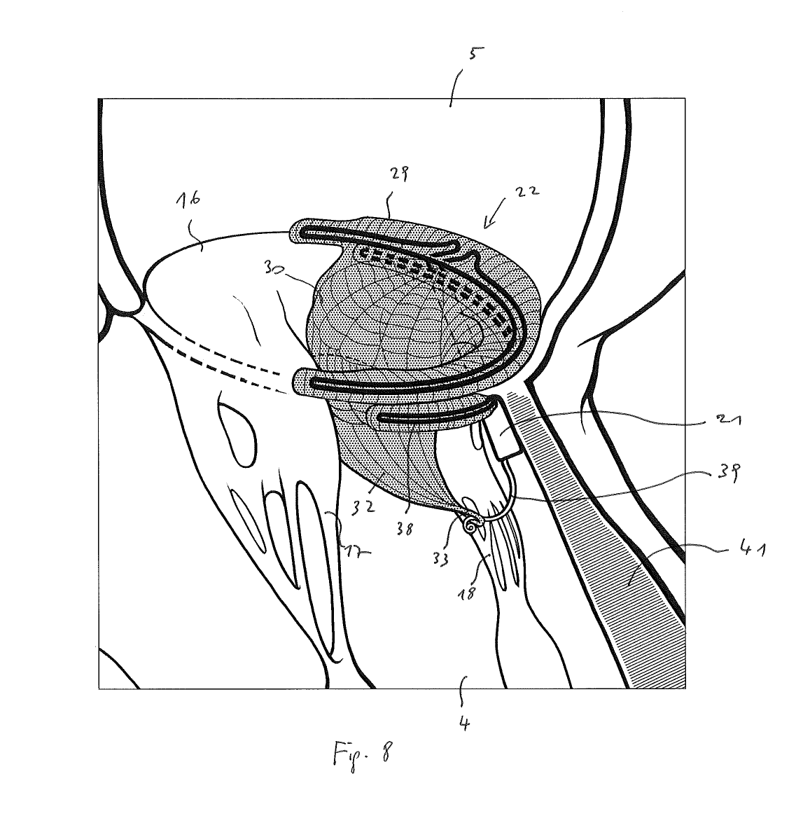

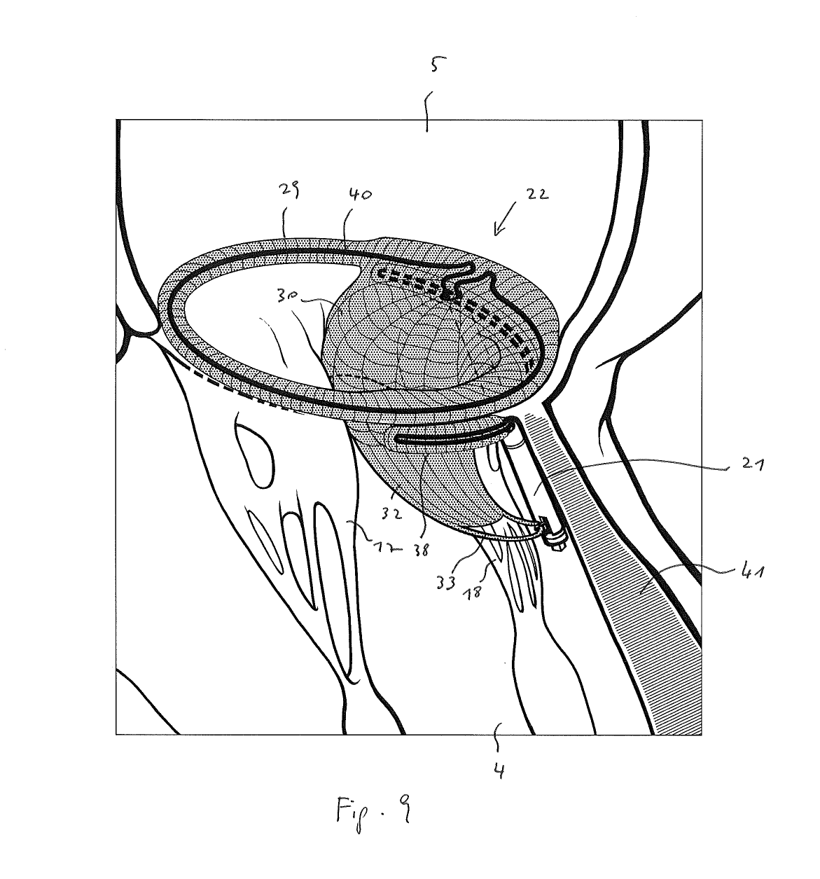

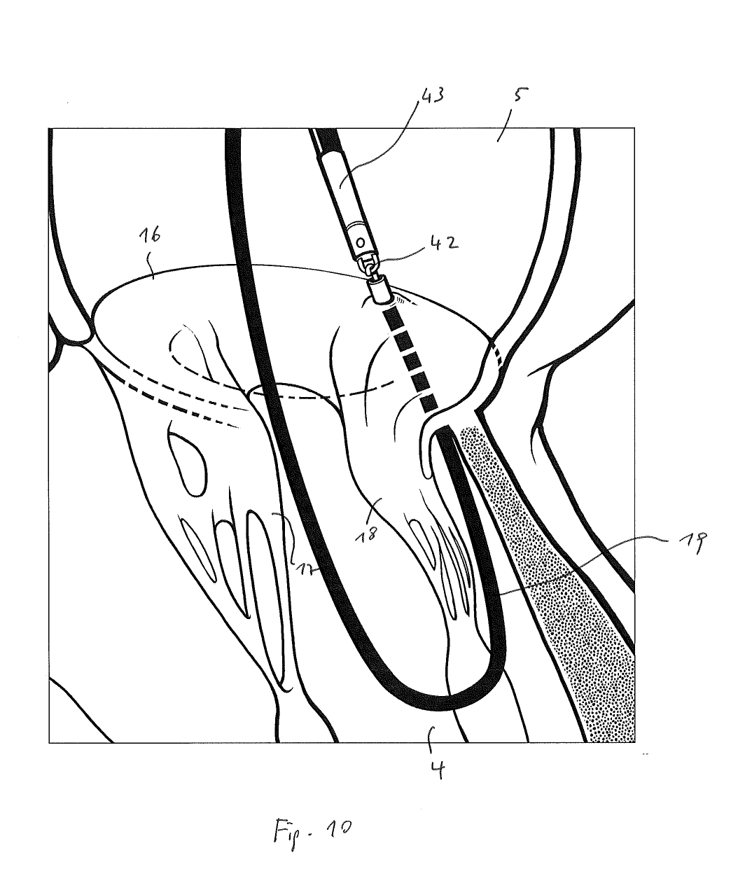

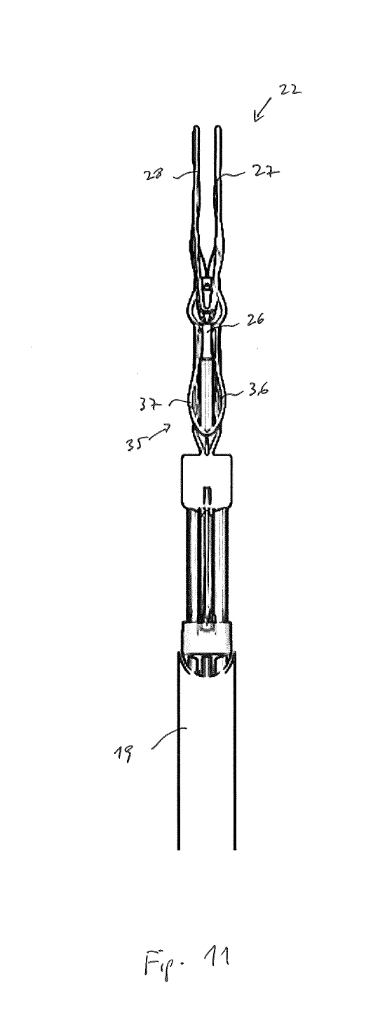

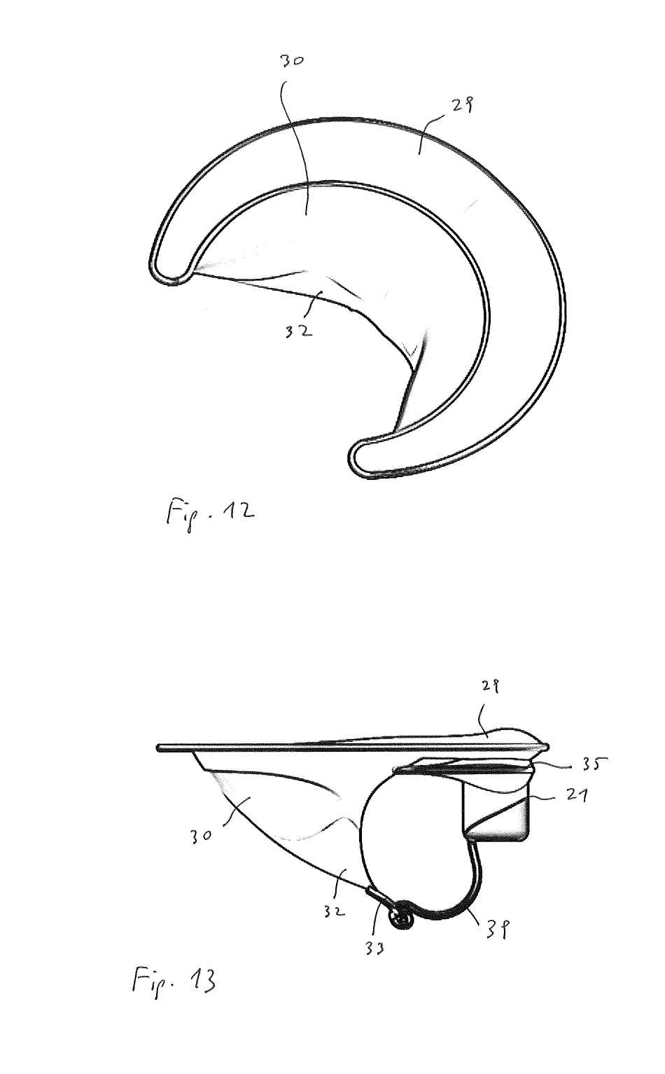

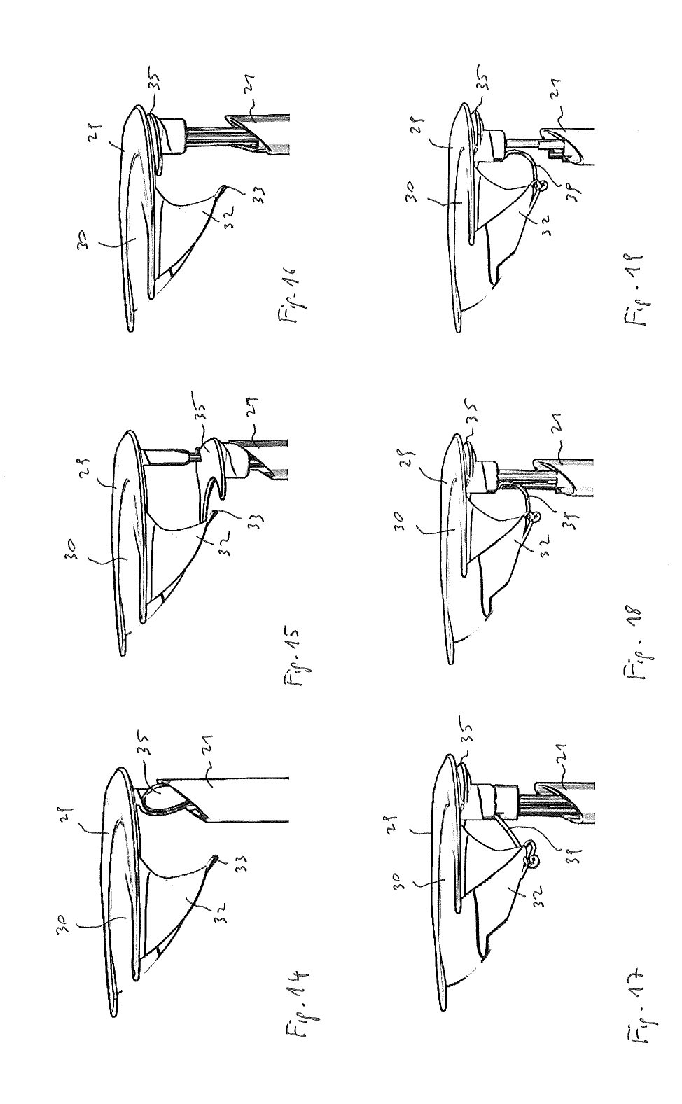

In FIG. 9 an alternative embodiment is illustrated, wherein the upper support element 22 comprises a circular wire 40 and a jacket 29 surrounding the circular wire 40, both extending along the entire length of the annulus 16. As with the embodiment according to FIGS. 1 to 8, the cavity of the upper support element 22 may be filled with a viscous fluid or a gel.

FIG. 10 shows an alternative way of advancing the catheter tip so as to penetrate the annulus 16 from below. A separate anchor 43 is introduced into the heart from above, i.e. form the left atrium, which is connected to the distal end of the catheter 19 by means of a hook mechanism 42, in order to be able to pull instead of push the catheter 19 to penetrate the annulus 16.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

1. An implant for improving coaptation of an atrioventricular valve, the atrioventricular valve having a native first leaflet, a native second leaflet and an annulus, the implant comprising a support structure and a flexible artificial leaflet structure mounted to the support structure and shaped to coapt with the native second leaflet.

2. The implant of claim 1, further comprising a tubular housing, wherein the support structure and the artificial leaflet structure are deployable from a first position, in which the support structure and the artificial leaflet structure are arranged within the tubular housing, into a second position, in which the artificial leaflet structure is deployed to coapt with the second native leaflet.

3. The implant of claim 1, wherein the artificial leaflet structure comprises a cavity.

4. The implant of claim 3, wherein the cavity comprises a closable opening for filling the cavity with a filling material.

5. The implant of claim 1, or 3, wherein the support structure comprises a cavity.

6. The implant of claim 5, wherein the cavity of the artificial leaflet structure and the cavity of the support structure are connected to each other to form a single cavity.

7. The implant of claim 3, wherein the cavity of the artificial leaflet structure and/or the cavity of the support structure are filled with a filling material, said filling material being selected from the group consisting of a fluid, an elastic solid, such as a foamed material, and a gel.

8. The implant of claim 1, wherein the support structure comprises an upper support element and a lower support element displaceable relative to each other so as to be able to squeeze a section of the annulus between them.

9. The implant of claim 8, wherein the upper support element is substantially U-shaped or circular.

10. The implant of claim 1, further comprising retention means connected to the support structure and the artificial leaflet for preventing prolapse of the artificial leaflet.

11. The implant of claim 1, wherein the atrioventricular valve is a mitral valve and the first native leaflet is a posterior leaflet of the mitral valve.

12. A method of improving coaptation of an atrioventricular valve, the atrioventricular valve having an annulus, a native first leaflet and a native second leaflet, the method comprising:
providing an implant comprising a support structure and a flexible artificial leaflet structure mounted to the support structure, the implant being arranged in a tubular housing,
advancing the tubular housing by means of a catheter through a body vessel of a patient into the heart,
deploying the implant from the tubular housing,
fixing the support structure relative to the annulus,
arranging the artificial leaflet structure adjacent the native first leaflet such that the artificial leaflet structure can coapt with the native second leaflet.

13. The method of claim 12, wherein the tubular housing is advanced into the heart by means of a catheter transatrially, transseptally, transfemorally or transapically.

14. The method of claim 12, wherein the step of fixing the support structure relative to the annulus comprises positioning an upper support element on a superior surface of the annulus and positioning a lower support element on an inferior surface of the annulus thereby clamping a section of the annulus between the upper support element and the lower support element.

15. The method of claim 12, wherein the step of fixing the support structure relative to the annulus comprises arranging the upper support element at least partially within the inner circumferential surface of the annulus and expanding the upper support element in a radial direction towards the inner circumferential surface of the annulus.

16. The method of claim 15, wherein the upper support element is expanded by filing a filling material into a cavity of the upper support element.

17. The method of claim 15, wherein the upper support element is expanded by expanding a filling material arranged in a cavity of the upper support element.

18. The method of claim 12, further comprising connecting the artificial leaflet to the support structure by the aid of retention means for preventing prolapse of the artificial leaflet.
